(19) United States
(12) Patent Application Publication
(10) Pub. No.: US 2023/0051063 A1
(43) Pub. Date: Feb. 16, 2023

(54) METHOD AND SYSTEM OF PULSE-ECHO ULTRASOUND IMAGING USING PSEUDO-RANDOM SPARSE ARRAYS

(71) Applicants: UNIVERSITY OF ROCHESTER, Rochester, NY (US); CARESTREAM HEALTH, INC., Rochester, NY (US)

(72) Inventors: Zeljko Ignjatovic, Rochester, NY (US); Jovan Mitrovic, Raleigh, NC (US); William J. Sehnert, Fairport, NY (US)

(21) Appl. No.: 17/817,211

(22) Filed: Aug. 3, 2022

Related U.S. Application Data
(60) Provisional application No. 63/260,091, filed on Aug. 9, 2021.

(51) Int. Cl. A61B 8/00; G01S 15/89; A61B 8/08

(52) U.S. Cl. CPC A61B 8/4494; G01S 15/8925; A61B 8/5207; G01S 15/8927; B06B 1/0607

(57) ABSTRACT

A method and system of pulse-echo ultrasound imaging by separating transducer elements of an ultrasound transducer array separate subsets, wherein the transducer elements in one subset performs a transmit operation only, and the transducer elements in the other subset perform an echo receive operation only; and grouping the transducer elements into groups of transducer elements based on subset, where each of the groups of transducer elements has the same probability of membership in either a transmit subset or a receive subset; and randomly concatenating the groups of transducer elements into a sparse array.

### RELATED APPLICATIONS

This application claims priority to U.S. provisional application No. 63/260,091, filed on Aug. 9, 2021, the contents of which are incorporated by reference in their entirety.

### FIELD

This application is related to the field of ultrasound imaging, in particular, the efficient production of high quality images through improved array design.

### BACKGROUND

A sparse array is an array where not all the channels are used for imaging. Depending on the configuration, this can mean that the arrays are treated as sparse during the transmit or receive event, or both. An unused channel is presumed to be turned off, and the associated hardware would not drain any power. As such, it is of utmost importance to find the optimal sparse array that would provide a good image quality, with the least amount of aberrations. Another benefit of the sparse array can be observed in allowing for power savings and hardware footprint reduction.

There is a need for improved pseudo-random sparse array design.

### SUMMARY

An aspect of the application is a method of pulse-echo ultrasound imaging comprising the steps of: separating transducer elements of an ultrasound transducer array into a first disjoint subset and a second disjoint subset, wherein the transducer elements in the first disjoint subset perform a transmit operation only, and wherein the transducer elements in the second disjoint subset perform an echo receive operation only; and grouping the transducer elements of the first disjoint subset and second disjoint subset into groups of transducer elements, wherein each group contains a plurality of transducer elements, and wherein each of the groups of transducer elements has the same probability of having transducer elements within each group in either the first disjoint subset (transmit) or the second disjoint subset (receive), or having transducer elements within each group in the first disjoint subset and second disjoint subset; and randomly concatenating the groups of transducer elements into a sparse array.

An aspect of the application is a method of pulse-echo ultrasound imaging comprising the steps of: separating transducer elements of an ultrasound transducer array into a first disjoint subset and a second disjoint subset, wherein the transducer elements in the first disjoint subset perform a transmit operation only, and wherein the transducer elements in the second disjoint subset perform an echo receive operation only; and grouping the transducer elements into a plurality of groups of transducer elements, wherein each group consists of two or more adjacent transducer elements and wherein each group consists of at least one transducer element of the first disjoint subset and second disjoint subset into groups of transducer elements; and randomly concatenating the groups of transducer elements into a sparse array.

In certain embodiments, the first disjoint subset and the second disjoint subset in the array each produce grating-lobe(s) and side-lobe(s), and the first disjoint subset and the second disjoint subset are separated to minimize peak magnitude of each of the grating-lobe(s) and side-lobes(s). In certain embodiments, the array has a point spread function, wherein the point spread function has a main lobe, and the first disjoint subset and the second disjoint subset are separated to spread the energy of the side-lobe(s) away from the main-lobe of the array's point spread function. In certain embodiments, the spread of the energy of the side-lobe(s) reduces the energy distribution close to the main-lobe's location to nearly zero, and the energy distribution then increases at the rate of at least +20 dB/dec when moving away from the main-lobe's location. In certain embodiments, the groups of transducer elements comprise two or more adjacent transducer elements.

In certain embodiments, half of the elements within a group are used for transmit, and half of the elements within a group are used for echo receive operation. In certain embodiments, the sparse array is formed by concatenating pairs of transducer elements selected at random from [1 0] and [0 1], wherein 1 represents an element within the pair used for transmit and 0 represents an element within the pair used for receive. In certain embodiments, the point spread function of the array resembles first-order blue noise. In certain embodiments, the sparse array is formed by concatenating quartets of transducer elements selected at random from [1 0 0 1] and [0 1 1 0], wherein 1 represents an element within the pair used for transmit and 0 represents an element within the pair used for receive. In certain embodiments, the point spread function of the array resembles second-order blue noise. In certain embodiments, the array is a one-dimensional array. In certain embodiments, the array is a two-dimensional array.

An aspect of the application is a system for pulse-echo ultrasound imaging comprising: a sparse array of transducer elements, wherein the transducer elements of the sparse array are ordered into groups of transducer elements, and wherein each of the groups of transducer elements has the same probability of membership in either a first disjoint subset (transmit) or a second disjoint subset (receive).

In certain embodiments, the groups comprise two or more adjacent transducer elements, and half of the elements within a group are used for transmit, and half of the elements within a group are used for echo receive operation, and the system further comprises: randomly concatenating the groups of transducer elements into a sparse array.

In certain embodiments, the sparse array is formed by concatenating pairs of transducer elements selected at random from [1 0] and [0 1], wherein 1 represents an element within the pair used for transmit and 0 represents an element within the pair used for receive.

In certain embodiments, the sparse array is formed by concatenating quartets of transducer elements selected at random from [1 0 0 1] and [0 1 1 0], wherein 1 represents an element within the pair used for transmit and 0 represents an element within the pair used for receive. In certain embodiments, the array is a one-dimensional array. In certain embodiments, the array is a two-dimensional array. In certain embodiments, the sparse array has a point spread function that resembles first-order blue noise. In certain embodiments, the sparse array has a point spread function that resembles second-order blue noise.

One of ordinary skill will understand that the differing embodiments disclosed in this application can all be used either independently or in combination with each other and there is no limitation implied on such combinations by the order or manner in which embodiments are disclosed.

### BRIEF DESCRIPTION OF THE DRAWINGS

While the present disclosure will now be described in detail, and it is done so in connection with the illustrative embodiments, it is not limited by the particular embodiments illustrated in the figures and the appended numbered paragraphs.

FIG. 1 illustrates an example of a block schematic showing the position of the T/R switch.

FIG. 2 illustrates a schematic of a T/R switch.

FIG. 3 illustrates an image of a beam pattern demonstrating the main, side and grating lobes.

FIG. 4 shows a graphical illustration of the proposed sparse array construction with the first-order (+20 dB/dec) blue-noise shaping of the side-lobe's energy distribution.

FIG. 5 shows a graphical illustration of the proposed sparse array construction with the second-order (+40 dB/dec) blue-noise shaping of the side-lobe's energy distribution.

FIG. 6 shows point spread function for a periodic, fully random, 1^{st} order blue noise random, and 2^{nd} order blue-noise random sparse array.

FIG. 7A-D shows comparison of the full and sparse aperture (random and periodic) array images.

FIG. 8A-D shows comparison of the full and sparse aperture (random and periodic) array images.

FIG. 9 shows larger cyst imaged with periodic sparse array.

### DETAILED DESCRIPTION OF THE INVENTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

A novel method for the sparse aperture array construction is presented and evaluated. The greatest benefit of choosing the random sparse array in the blue noise manner is the fact that the contrast remains strong, while not a lot of noise is added, especially not in the focal area. Furthermore, it outperforms the periodic and the white noise random array overall. The power savings that a high-quality sparse array allows are significant, and, although evaluated for a 1-D array, this method of aperture choice can be easily extended to the 2-D arrays as well.

In order to understand how to minimize the hardware footprint, an understanding of how the transmit/receive (T/R) switch works is necessary.

T/R Switch

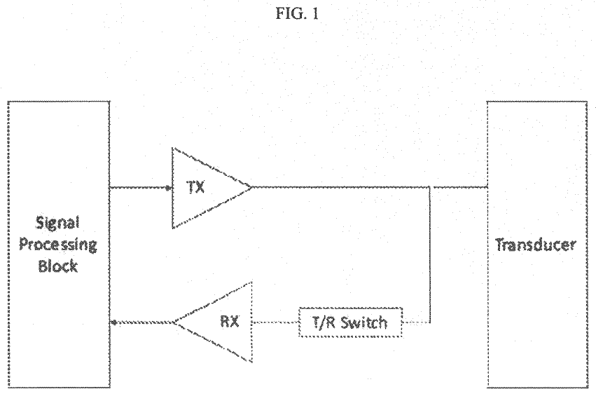

A T/R switch is a specialized circuit used for hardware that allows both transmitting and receiving to be performed through the same channel, but not at the same time instance. This is the case for many of the RF circuits, and, in a way, ultrasound acquisition is architecturally very similar to the architecture of the RF circuits. The position of this circuit in the signal acquisition chain can be observed in the block diagram of the ultrasound imaging AFE presented in FIG. 1.

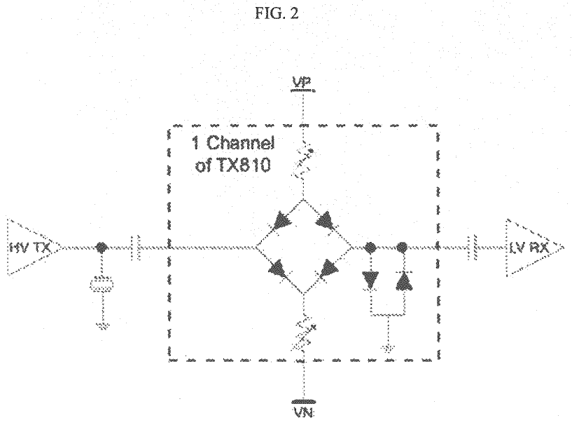

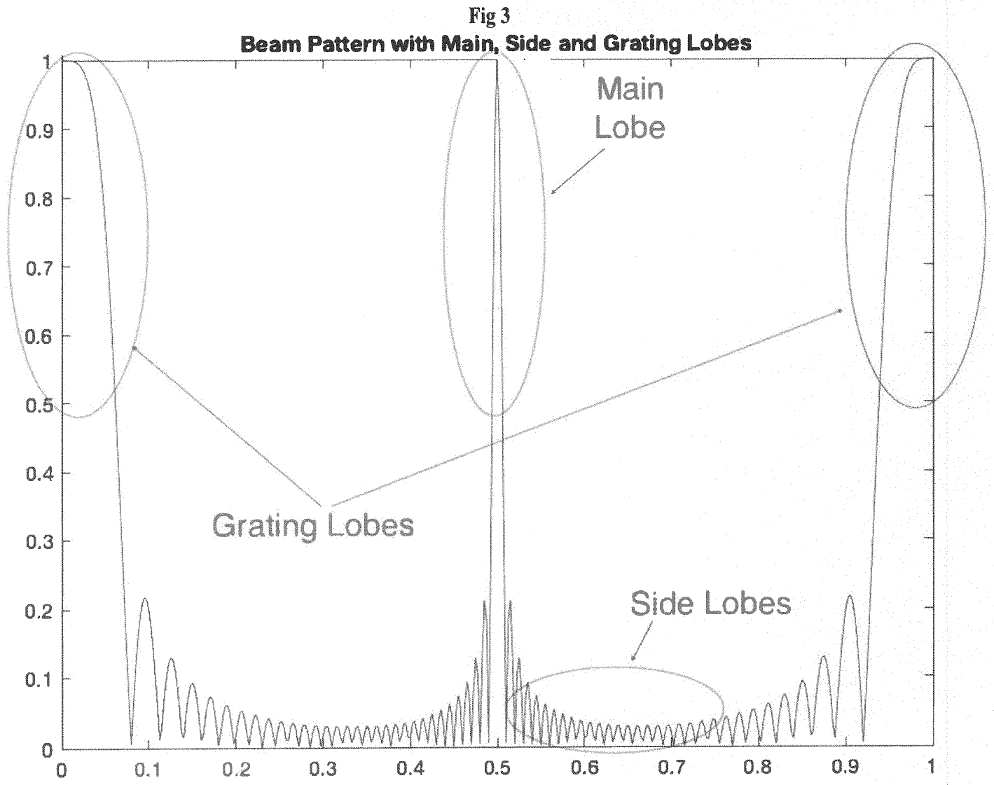

From FIG. 1, it can be observed that the T/R switch should not interfere with the transmitting event; rather, its purpose in ultrasound imaging is to limit (ideally, completely eliminate) the voltage that the rece1vmg end sees during the transmitting event. Specifically, a transmit event is usually characterized by a 1001/pulse; flowing towards the transducer. As the receiving end consists of many amplifiers and filters designed to work with small signals (order of magnitude of l0rnV), an input of 100V would burn these circuits even with the circuits turned off. In addition. the T/R Switch should conduct the signal during the receive event with as little noise as possible. These circuits in the design do not need to be very complicated. However they do consume a somewhat significant portion of power when high voltage is present. A schematic of one of the T/R Switches available on the market is shown in FIG. 2.

Ultimately, what the T/R switch allows the ultrasound imaging system to do is to treat every transducer element as the transmitter during the transmit event, and then as a receiver during the receive event. This allows the most coherent and uniform wavefront to be delivered, and the most power to be delivered and received which is very important for the efficiency of the imaging.

Sparse Array Power Savings Optimization

From the discussion about the T/R switches, it is clear that the most hardware footprint savings will be achieved if the T/R switches are removed completely. However, if the switches were to be removed, a channel can't perform in both transmit and receive mode, and a choice needs to be made during the hardware design phase over which channels will be transmit-only, and which ones receive-only.

Method

An aspect of the application is a method of pulse-echo ultrasound imaging comprising the steps of: separating transducer elements of an ultrasound transducer array into a first disjoint subset and a second disjoint subset, wherein the transducer elements in the first disjoint subset perform a transmit operation only, and wherein the transducer elements in the second disjoint subset perform an echo receive operation only; and grouping the transducer elements of the first disjoint subset and second disjoint subset into groups of transducer elements, and wherein each of the groups of transducer elements has the same probability of membership in either a first disjoint subset (transmit) or a second disjoint subset (receive); and randomly concatenating the groups of transducer elements into a sparse array.

Another aspect of the application is a method of pulse-echo ultrasound imaging comprising the steps of: separating transducer elements of an ultrasound transducer array into a first disjoint subset and a second disjoint subset, wherein the transducer elements in the first disjoint subset perform a transmit operation only, and wherein the transducer elements in the second disjoint subset perform an echo receive operation only; and grouping the transducer elements into a plurality of groups of transducer elements, wherein each group consists of two or more adjacent transducer elements and wherein each group consists of at least one transducer element of the first disjoint subset and second disjoint subset into groups of transducer elements; and randomly concatenating the groups of transducer elements into a sparse array.

Pulse-echo ultrasound involves the transmission of a short pulse of sound, followed by a period in which the transducer “listens” for the returning echoes. There are several properties of ultrasound that are useful in clinical cardiology. Since ultrasound is a mechanical wave in a longitudinal direction, it is transmitted in a straight line and it can be focused. These waves obey laws of reflection and refraction. Since small objects in the human body will reflect ultrasound, it is possible to collect the reflected data and compose a picture of these objects to further characterize them. The major drawback of ultrasound is the fact that it cannot be transmitted through a gaseous medium (like air or lung tissue), in clinical echo certain windows are used to image the heart and avoid the lungs. As ultrasound transverses tissue, its energy decreases. This is called attenuation and is more pronounced in tissue with less density (like lung). There are seven parameters that describe ultrasound waves. The period of an ultrasound wave is the time that is required to capture one cycle, i.e., the time from the beginning of one cycle till the beginning of the next cycle. The units of period is time and typical values in echo is 0.1 to 0.5 microsecond. Period of ultrasound is determined by the source and cannot be changed by the sonographer. Frequency is the inverse of the period and is defined by a number of events that occur per unit time. The units of frequency is 1/sec or Hertz (Hz). Since f=1/P, it is also determined by the source and cannot be changed. Amplitude is an important parameter and is concerned with the strength of the ultrasound beam. It is defined as the difference between the peak value and the average value of the waveform. It is expressed in decibels or dB, which is a logarithmic scale. It can be changed by a sonographer. Amplitude decreases as the ultrasound moves through tissue, this is called attenuation. Amplitude decreases usually by 1 dB per 1 MHz per 1 centimeter traveled. For example, if we have a 5 MHz probe and the target is located at 12 cm (24 cm total distance), then the amplitude attenuation will be 1 dB×5 MHz×24 cm=120 dB which nearly 6000 fold decrease.

Power of ultrasound is defined as the rate of energy transfer and is measured in Watts. It is determined by the sound source and it decreases as the beam propagated through the body. Intensity of the ultrasound beam is defined as the concentration of energy in the beam. Intensity=Power/beam area=(amplitude)²/beam area, thus it is measured in Watts per cm². It is the key variable in ultrasound safety. Intensity also decreases as the ultrasound propagates through tissue. Wavelength is defined as the length of a single cycle. It is measured in the units of length. It is determined by both the source and the medium. Wavelength cannot be changed by the sonographer. It influences the longitudinal image resolution and thus effect image quality. Typical values of wavelength are 0.1-0.8 mm. Wavelength (mm)=Propagation speed in tissue (mm/microsecond)/frequency (MHz). High frequency means short wavelength and vice versa.

Propagation speed in human soft tissue is on average 1540 m/s. It is defines as to how fast the ultrasound can travel through that tissue. It is determined by the medium only and is related to the density and the stiffness of the tissue in question. Density of the medium is related to its weight and the stiffness of the medium is related to its “squishability”. As the medium becomes more dense, the slower is speed of ultrasound in that medium (inverse relationship). The stiffer the tissue, the faster will the ultrasound travel in that medium (direct relationship). There are tables where one can look up the velocity of sound in individual tissues. Range equation—since ultrasound systems measure the time of flight and the average speed of ultrasound in soft tissue is known (1540 m/s), then we can calculate the distance of the object location. Distance to boundary (mm)=go-return time (microsecond)×speed (mm/microsecond)/2. So far we have defined the ultrasound variables and parameters. In the next section will talk more about pulsed ultrasound. Pulse Duration is defined as the time that the pulse is on. It is determined by the number of cycles and the period of each cycle. In clinical imaging, a pulse is comprised of 2-4 cycles and the pulse duration is usually between 0.5 to 3 microseconds. Pulse duration does not change with depth, thus it cannot be changed by the sonographer. Pulse Duration (msec)=# of cycles×period (msec). Since Wavelength (mm)=Propagation speed in tissue (mm/microsecond)/frequency (MHz), this can be rewritten as 1/frequency=wavelength/propagation speed. And since period=1/frequency, then the Pulse Duration=(# of cycles×wavelength)/Propagation speed.

Pulse Repetition Period or PRP is the time between the onset of one pulse till the onset of the next pulse. Again, it is measured in units of time. This parameter includes the time the pulse is “on” and the listening time when the ultrasound machine is “off”. It can be changed by the sonographer by varying the depth to which the signal is send. Since the Pulse Duration time is not changed, what is changed is the listening or the “dead time”. PRP=13 microseconds×the depth of view (cm). It follows from this equation that the deeper is the target, the longer is the PRP. The typical values of PRP in clinical echo are form 100 microseconds to 1 millisecond. A related parameter to PRP is the Pulse Repetition Frequency or PRF. PRP and PRF are reciprocal to each other. PRF is the number of pulses that occur in 1 second. This parameter is not related to the frequency of ultrasound. PRF can be altered by changing the depth of imaging. It is measured in Hertz (Hz). PRF=77,000/depth of view (cm). As evident from the equation, as the location of the target gets further away, the PRF decreases. PRF is related to frame rate or sampling rate of the ultrasound. I would like to talk about Duty Factor (DF) here. This parameter is related to ultrasound bioeffects, but since it is also related to pulsed ultrasound it is reasonable to introduce it in this section. DF is defined as a percent of time that the ultrasound system is on while transmitting a pulse. DF=pulse duration (sec)/pulse repetition period (sec)×100. It has units of % and ranges from 0 (the system is off) to 100 (the system is on continuously). Typical valued of DF in clinical imaging are 0.1% to 1% (usually closer to 0), thus the machine is mostly listening during clinical imaging. Another interesting point to note is the fact that since the sonographer changes the PRF by changing the depth, they indirectly change the duty factor. And lastly, one must realize that an anatomic image cannot be created with a continuous wave ultrasound. Since one must listen for the return signal to make an image, a clinical echo machine must use pulsed signal with DF between 0.1 and 1%.

Spatial Pulse Length is the distance that the pulse occupies in space, from the beginning of one pulse till the end of that same pulse. It is measured in units of distance with typical values from 0.1 to 1 mm. SPL (mm)=# cycles×wavelength (mm). Axial or longitudinal resolution (image quality) is related to SPL. Axial resolution=SPL/2=(# cycles×wavelength)/2.

The energy of ultrasound decreases (attenuation) as it travels through tissue. The stronger the initial intensity or amplitude of the beam, the faster it attenuates. Standard instrument output is ˜65 dB. So for a 10 MHz transducer, the maximum penetration would be as follows: 1 dB/cm/MHz×10 MHz×(2×max depth)=65 dB. Max depth=65/20=3.25 cm. If a 3.5 MHz transducer is used and the same formula is applied for max depth, will get Max depth=65/7=9.3 cm. Attenuation of ultrasound in soft tissue depends on the initial frequency of the ultrasound and the distance it has to travel. As in the example above, in soft tissue the greater the frequency the higher is the attenuation. So a deeper image can be obtained with a lower frequency transducer. The further into the tissue the ultrasound travels, the higher the attenuation is, so it is ultimately the limiting factor as to how deep to image clinically relevant structures.

There are three components of interaction of ultrasound with the tissue medium: absorption, scattering, and reflection. Absorption of ultrasound by tissue implies loss of energy that is converted to heat. The highest attenuation (loss of energy) is seen in air, the lowest is seen in water. Reflection is the process were propagating ultrasound energy strikes a boundary between two media (i.e., the RV free wall in the parasternal long axis) and part of this energy returns to the transducer. If the reflector is very smooth and the ultrasound strikes it at 90 degree angle (perpendicular), then the reflection is strong and called specular. If the incidence is not 90 degree, then specular reflectors are not well seen. Another instance when specular reflection is produced is when the wavelength is much smaller than the irregularities of the media/media boundary. Diffuse or Backscatter reflections are produced when the ultrasound returning toward the transducer is disorganized. This occurs when the ultrasound wavelength is similar size to the irregularities of the media/media boundary. When the ultrasound wavelength is larger than the irregularities of the boundary, the ultrasound is chaotically redirected in all directions or scatters. If the reflector is much smaller than the wavelength of the ultrasound, the ultrasound is uniformly scattered in all directions and this is called Rayleigh scattering. Red blood cell would be an example of Rayleigh scatterer. Rayleigh scattering is related to wavelength to 4th power. Backscatter is what produces the relevant medical imaging.

Impedance (Z) is an important concept and it is related to reflection of ultrasound energy. It is calculated and is not measured directly. The higher the difference of the acoustic impedance between two media, the more significant is the reflection of the ultrasound. That is why a coupling gel is used between the ultrasound transducer and the skin. By using the gel, the impedance is decreased and the ultrasound is allowed to penetrate into the tissue. Otherwise, the impedance between skin/transducer is so high that all the energy will be reflected and no image will be produced. Reflection occurs only when the acoustic impedance of one media is different from acoustic impedance of the second media at the boundary. If the ultrasound hits the reflector at 90 degrees (normal incidence), then depending on the impedances at the boundary the % reflection=((Z2−Z1)/(Z2+Z1))². Then transmission is 1−% reflection. Physics of oblique incidence is complex and reflection/transmission may or may not occur. The incident intensity is equal to the sum of the transmitted and reflected intensities.

Refraction is simply transmission of the ultrasound with a bend. This occurs when we have an oblique incidence and different propagation speed from one media to the next. The physics of the refraction is described by Snell's law. Sine (transmission angle)/sine (incident angle)=propagation speed 2/propagation speed 1. Axial resolution (ability to differentiate objects that are located along the imaging beam axis) is related to spatial pulse length. The smaller the axial resolution length, the better the system is and it can resolve structures that are closer together. Thus, the shorter the pulse length, the better picture quality. Current transducers are designed with the minimum number of cycle per pulse to optimize image quality. The primary determinant of axial resolution is the transducer frequency. Axial resolution (mm)=0.77×# cycles/frequency (MHz). One must remember that attenuation is also dependent on the transducer frequency, thus a tradeoff must be reached. Lateral resolution is the minimum distance that can be imaged between two objects that are located side to side or perpendicular to the beam axis. Again, the smaller the number the more accurate is the image. Since the beam diameter varies with depth, the lateral resolution will vary with depth as well. The lateral resolution is best at the beam focus (near zone length) as will discuss later when will talk about the transducers. Lateral resolution is usually worse than axial resolution because the pulse length is usually smaller compared to the pulse width. Temporal resolution implies how fast the frame rate is. FR=77000/(# cycles/sector×depth). Thus, frame rate is limited by the frequency of ultrasound and the imaging depth. The larger the depth, the slower the FR is and worse temporal resolution. The higher the frequency is, the higher is the FR and the temporal resolution improves. Sonographer can do several things to improve the temporal resolution: images at shallow depth, decrease the #cycles by using multifocusing, decrease the sector size, lower the line density. However, one can realize quickly that some of these manipulations will degrade image quality. And this is in fact correct: improving temporal resolution often degrades image quality.

The current transducers became available after the discovery that some materials can change shape very quickly or vibrate with the application of direct current. As important is the fact that these materials can in turn produce electricity as they change shape from an external energy input (i.e., from the reflected ultrasound beam). This effect of vibration form an application of alternative current is called a piezoelectric effect (PZT). Many materials exist in nature that exhibit piezoelectric effect. Commercial transducers employ ceramics like barium titanate or lead zirconate titanate. The transducer usually consists of many PZT crystals that are arranged next to each other and are connected electronically. The frequency of the transducer depends on the thickness of these crystals, in medical imaging it ranges 2-8 MHz. An ultrasound pulse is created by applying alternative current to these crystals for a short time period. Afterwards, the system “listens” and generates voltage from the crystal vibrations that come from the returning ultrasound. An important part of the transducer is the backing material that is placed behind the PZT, it is designed to maximally shorten the time the PZT crystal vibrates after the current input is gone also known as ringing response. By decreasing the ringdown time, one decreases the pulse length and improves the axial resolution. In addition, the backing material decreases the amount of ultrasound energy that is directed backwards and laterally.

In front of the PZT, several matching layers are placed to decrease the difference in the impedance between the PZT and the patient's skin. This increases in efficiency of ultrasound transfer and decrease the amount of energy that is reflected from the patient. Let us talk about the shape of the ultrasound beam. Since there are many PZT crystals that are connected electronically, the beam shape can be adjusted to optimize image resolution. The beam is cylindrical in shape as it exits the transducer, eventually it diverges and becomes more conical. The cylindrical (or proximal) part of the beam is referred to as near filed or Freznel zone. The image quality and resolution is best at the focal depth that can be determined by Focal depth=(Transducer Diameter)×frequency/4. When the ultrasound beam diverges, it is called the far field.

One would state that the best images are acquired using a large diameter transducer with high frequency. However, high frequency transducers have significant attenuation issues. In addition, larger diameter transducers are impractical to use because the imaging windows are small. The way around these problems is electronic focusing with either an acoustic lens or by arranging the PZT crystals in a concave shape.

In clinical imaging, the ultrasound beam is electronically focused as well as it is steered. This became possible after phased array technology was invented. By applying electrical current in a differential manner and adjusting the timing of individual PZT excitation, the beam can travel in an arch producing a two-dimensional image. If one applies electricity in a differential manner from outside inward to the center of the transducer, differential focusing can be produced resulting in a dynamic transmit focusing process.

In real time 3D imaging, the PZT elements need to be arranged in a 2D matrix. Each PZT element represents a scan line, by combining all the data, a 3D set is reconstructed. For example, with a matrix of 128 by 128 PZT elements, one can generate over 16 thousand scan lines. With careful timing for individual excitation, a pyramidal volumetric data set is created. When imaged several times per minute (>20), a real time image is achieved.

Image production is a complex process. Echo instrumentation must generate and transmit the ultrasound and receive the data. Then the data needs to be amplified, filtered and processed. Eventually the final result needs to be displayed for the clinician to view the ultrasound information. As the first step in data processing, the returning ultrasound signals need to be converted to voltage. Since their amplitude is usually low, they need to be amplified. The ultrasound signal usually is out of phase so it needs to be realigned in time. At this point one has the raw frequency (RF) data, which is usually high frequency with larger variability in amplitudes and it has background noise. The next step is filtering and mathematical manipulations (logarithmic compression, etc.) to render this data for further processing. At this stage one has sinusoidal data in polar coordinates with distance and an angle attached to each data point. This information needs to be converted to Cartesian coordinate data using fast Fourier transform functions. Once at this stage, the ultrasound data can be converted to analog signal for video display and interpretation. Image display has evolved substantially in clinical ultrasound. Currently, 2D and real time 3D display of ultrasound date is utilized. Without going into complexities of physics that are involved in translating RF data into what we see every day when one reads echo, the following section will provide the basic knowledge of image display. If one can imagine a rod that is imaged and displayed on an oscilloscope, it would look like a bright spot. Displaying it as a function of amplitude (how high is the return signal) is called A-mode. If one converts the amplitude signal into brightness (the higher the amplitude the brighter the dot is), then this imaging display is called B-mode. Using B mode data, once can scan the rod multiple times and then display the intensity and the location of the rod with respect to time. This is called M-mode display. Using B-mode scanning in a sector created a 2D representation of anatomical structures in motion.

Second Harmonic is an important concept that is used today for image production. The basis for this is that fact that as ultrasound travels through tissue, it has a non-linear behavior and some of its energy is converted to frequency that is doubled (or second harmonic) from the initial frequency that is used (or fundamental frequency). There are several parameters that make second harmonic imaging preferential. Since it is produced by the tissue, the deeper the target the more second harmonic frequency is returned. As the ultrasound beam travels through tissue, new frequencies appear that can be interrogated. Second harmonic data gets less distortion, thus it produces better picture. Also, the second harmonic is strongest in the center of the beam, thus it has less side lobe artifacts. At the chest wall the fundamental frequency gets the worst hit due to issues that we have discussed (reflection, attenuation)—if one can eliminate the fundamental frequency data then these artifacts will not be processed. One concept of eliminating fundamental frequency data is called pulse inversion technology. The transducer sends out two fundamental frequency pulses of the same amplitude but of different phase. As these pulses are reflected back to the transducer, because of the different phase they cancel each other out (destructive interference) and what is left is the second harmonic frequency data which is selectively amplified and used to generate an image.

Doppler Effect is a change in frequency of sound as a result of motion between the source of ultrasound and the receiver. Greater velocity creates a larger shift in ultrasound frequency. An example of a moving object in cardiac ultrasound is red blood cells. Typical values for Doppler shift is 20 Hz to 20 kHz, thus comparing to the fundamental frequency, the Doppler shift is small. Since it “rides” on top of the much larger frequency (i.e., 5 MHz), the process of extracting this data is termed demodulation. Doppler shift=(2×reflector speed×incident frequency×cosine (angle))/propagation speed. There are two important concepts that must be emphasized. First, the Doppler shift is highly angle dependent. Since cosine (90)=0 and cosine (0)=1, then the most true velocity will be measured when the ultrasound beam is parallel to the axis of motion of the reflector. At perpendicular axis, the measured shift should be 0, however usually some velocity would be measured since not all red blood cells would be moving at 90 degree angle. The other concept is the direction of the motion of the reflector. When the reflector is moving away from the source of the ultrasound, the shift is negative, and when the reflector is moving towards the source of ultrasound the shift is positive. Continuous wave (CW) Doppler required 2 separate crystals, one that constantly transmits, and one that constantly receives data. There is no damping using this mode of imaging. One can measure very high velocities (i.e., velocities of aortic stenosis or mitral regurgitation). The advantage of CW is high sensitivity and ease of detecting very small Doppler shifts. The disadvantage of CW is the fact that echoes arise from the entire length of the beam and they overlap between transmit and receive beams. Thus, one cannot determine where in the body the highest velocity is coming from—range ambiguity.

Pulsed wave (PW) Doppler requires only one crystal. It alternates between transmitting and receiving data. The transducer “listens” for the data at a certain time only, since the sampling volume is coming from the location that is selected by the sonographer (i.e., the velocity at the LVOT or at the tips of the mitral valve). This is called range resolution. The major disadvantage of PW Doppler is aliasing. In PW mode, the transducer has to sample a certain frequency at least twice to resolve it with certainty. This put a limit on the max velocity that it can resolve with accuracy. 2×Doppler frequency (Nyquist)=PRF. If the velocity is greater than the sampling rate/2, aliasing is produced. The following maneuvers can be performed to eliminate aliasing: change the Nyquist limit (change the scale), select a lower frequency transducer, select a view with a shallower sample volume.

Color Flow Doppler uses pulsed Doppler technique. The velocity data is encoded in color, and it reports mean velocities. Since it is a pulsed Doppler technique, it is subject to range resolution and aliasing. Color data is extremely complex and consumes significant computational resources, thus several assumptions are made to speed up this process. Returned echo frequencies are compared to a predetermined threshold to decide whether this is a 2D image vs Doppler shift. Once the computer decides that the frequency is low enough to be a Doppler shift data, repetitive sampling determines the mean velocity and variance. Then a color is assigned using a color look-up table rather than doing a discrete Fourier transform for each data point. Velocities that move toward the transducer are encoded in red, velocities that move away are encoded in blue. One must remember that the color jets on echo are not equal to the regurgitant flow for a number of reasons. The regurgitant flow is a three dimensional structure with jet momentum being the primary determinant of jet size. This parameter is effected by the jet velocity as well as flow rate. Blood pressure will affect the velocity and thus the regurgitant flow. Chamber constraints will have an effect on the appearance of the color jet, especially eccentric jets. Lastly, the settings of the echo machine will have an effect on how the color flow jet appears on the screen.

System

An aspect of the application is a system for pulse-echo ultrasound imaging comprising: a sparse array of transducer elements, wherein the transducer elements of the sparse array are ordered into groups of transducer elements, and wherein each of the groups of transducer elements has the same probability of membership in either a first disjoint subset (transmit) or a second disjoint subset (receive), and randomly concatenating the groups of transducer elements into a sparse array.

An ultrasound transducer, or probe, emits sound waves in discrete bundles or pulses into the tissue of interest. On encountering a tissue, a portion of the waves is reflected back to the transducer. The fraction of returning waves depends on density and size of the tissue examined. The depth of tissue is determined by the time required for pulse emission and return. Thus, by integrating the number of returning pulses and the time required for return, a B-mode, or gray-scale image may be created. The time for wave reflection decreases with higher ultrasound probe frequencies. Transducer probes with higher frequencies image superficial tissues better than probes with lower frequencies, but lose depth imaging because of attenuation of the returning emitted pulses.

Ultrasound transducers consist of piezoelectric crystals that emit and receive high-frequency sound waves by interconverting electrical and mechanical energy. Transducer selection is important to the success of ultrasound-guided regional anesthesia procedures. High-frequency sound waves provide the best resolution but will not penetrate far into tissue. The frequency range is therefore chosen to be the highest that will allow adequate insonation of the entire depth of field. A low-frequency transducer can be used to image large nerves that lie deep, such as the cords of the brachial plexus that surround the second part of the axillary artery or the proximal sciatic nerve in the gluteal region.

The footprint size (i.e., the length of the active face transducer that contacts the skin) is chosen to provide a broad enough view of the structures of interest. As a general rule, the footprint should be at least as large as the anticipated depth of field. A square or landscape view is better than a keyhole view (i.e., depth greater than footprint) for guidance. As a rule of thumb, for in-plane technique, every millimeter of the footprint is approximately a millimeter of guidance

Linear-array transducers generally have a higher scan-line density than curved arrays and therefore produce the best image quality. Images from linear arrays are usually displayed in a rectangular format. When a linear transducer is needed but space at the site of block is limited by anatomic structures such as adjacent bone, a compact linear (hockey stick) transducer that has a smaller footprint can be very useful. Curved arrays provide a broad field of view for a given footprint size and are generally used when space is limited (e.g., infraclavicular region). Curved probes are easier to rock and produce images in sector format.

A strong case against the usage of the sparse arrays is often made because by reducing the amount of transmitting elements, a lower powered wave with less control over the focusing is propagated through the tissue. The same can be said for the receiving elements, where reducing the number of the elements and/or disrupting the geometry can yield artifacts, as already discussed. Balancing these two, a solution where half of the aperture transmits, and the other half receives the reflected wave is the best solution.

The power savings of this method are twofold: first, there is no power dissipation on the T/R switches during the receive event. Second, fewer transmitting elements, will be excited, which will require a lower amount of power. Additional power savings are possible and can be based on the compressed sensing framework. Embodiments of the method are discussed below.

The present application is further illustrated by the following examples that should not be construed as limiting. The contents of all references, patents, and published patent applications cited throughout this application, as well as the Figures and Tables, are incorporated herein by reference

### EXAMPLES

Simulation Configuration

The simulation results provided were made in Field 2. A linear array res-onating at 3.5M Hz with 192 elements was used, out of which 64 were active at each transmit/receive event. Transmit focus in every image was set at 60 mm depth, while the dynamic focusing was applied during receiving. 100 steered ray lines were taken, and the images were formed through standard beamforming methods.

The same simulation scheme was used for all the acquisitions. When simulating sparse arrays, a vector of l's and O's was generated in a manner specific to the sparse array type and applied as a transmit aperture. This vector would describe the physical connection of the array elements to the transmit end (i.e. 1 means that the element operates in transmit mode, and 0 means that the element is turned off during the transmit mode). The complement of this vector was then applied as a receive aperture in the same way. As complementary vectors do not intersect, T/R switches can be considered removed, along with the hardware bulk on the receiving end for the transmit-only elements 1 and vice-versa.

Metrics

The following section uses several metrics to evaluate the optimal sparse array from the imaging quality perspective. Some of the metrics, namely CR, CNR, and GCNR, will be evaluated in the same manner here.

However, in order to fully characterize an array, the description of its point spread function (PSF) is important. A PSF represents the response of the imaging system to a point source or, more accurately, it represents an impulse response of an imaging system.

From the PSF images, the levels of the main, side, and grating lobes can be extracted. The positioning of these lobes is shown in FIG. 3. Because the beam envelope is normalized to the maximum value the peak main lobe will always be at OdB. Sparse arrays introduce strong side or grating lobe, which are the main source of artifacts on the images. The lower the grating/side lobes relative to the main lobe, the better the image quality will be. This is why the peak value of the side/grating lobes will be presented, in dB relative to the peak main lobe.

Finding the Optimal Sparse Array

As one of the most crucial elements of sparse array imaging represents the choice of the sparse array itself, it is very important to determine the optimal configuration under which the output image looks as close to the full-aperture array as possible. Noticing a pattern in the way that the sparse array is constructed can ease the understanding and analysis of the given sparse array, and it can also ease the process of replication and construction. For example, two usual sparse array configurations are periodic and fully random sparse array (where fully random indicates that each transducer element is chosen at random with equal probability to serve either as a transmit or receive element). A periodic sparse array yields high grating lobes, while a fully random sparse array redistributes this energy from the grating lobes into the side lobes.

The reason why a fully random sparse array acts in the described way lies in the fact that every element has the same probability to be either on or off during transmit or receive (i.e. have the apodization of either 1 or 0). This means that there can be multiple adjacent elements that are on and multiple adjacent elements that are off. Although this gels rid of the periodicity, it can severely underperform as compared to the full-aperture array, especially if the region of interest on the image falls directly below the numerous adjacent elements that are off during transmit phase.

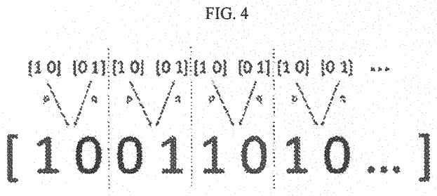

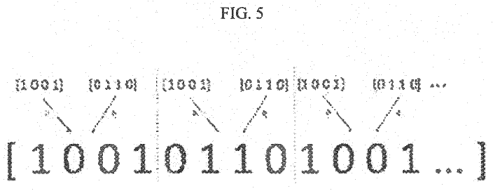

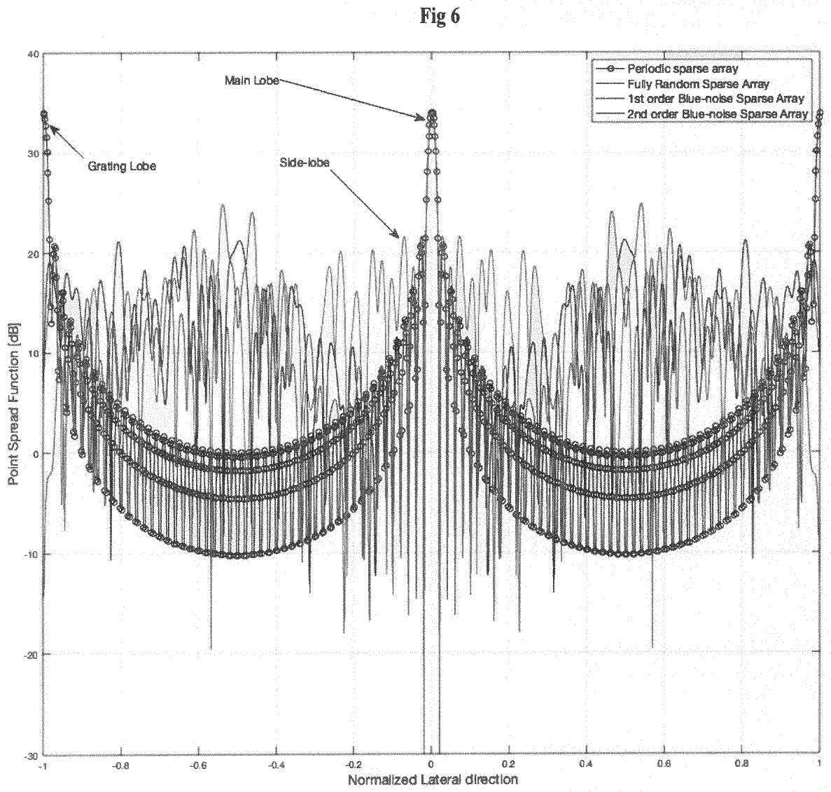

This patent application discloses a novel approach of forming random sparse arrays, in which the grating lobes are significantly reduced, as in the case of a fully random array, but not at the expense of much higher side lobes, and in which no more than two adjacent elements serve as transmit only. The way to obtain this performance is by introducing some constraints upon the randomness of the sparse array: instead of each element having the same probability of being either used for transmit or receive operation: a group of elements should be described by this probability. In order to reduce the periodicity as much as possible, groups consisting of two (or more) adjacent elements1 where half of the elements within a group are used for transmit and the other half for echo receive operation are formed and then randomly con-catenated into an array. A graphical representation of the process of forming an example sparse random array that not only reduces peak magnitudes of both the side-lobes and grating-lobes but also shapes the side-lobe's energy away from the main-lobe can be seen in FIG. 4. In this particular example, the sparse array is formed by concatenating pairs of transducer elements selected at random from [1 0] or [0 1], where 1 represents an element within the pair used for transmit and 0 represents the receive element. As opposed to the fully random sparse array, where the choice of the elements resem-bles white noise, the choice of the elements for the proposed array resembles the first-order blue noise (i.e., an array whose point spread function exhibits not only spreading of both side-lobes' and grating lobes' energy but also a shaping of the energy components away from the main-lobe at +20 dB/dec as seen in FIG. 6. In another example, a random sparse array is formed by concatenating groups of four transducer elements selected at random from [1 0 0 1] or [0 1 1 0] as can be seen in can be seen in FIG. 5. As opposed to the fully random sparse array, where the choice of the elements resembles white noise, the choice of the elements in this example resembles the second—order blue noise (i.e., an array whose point spread function exhibits not only spreading of both side-lobes' and grating lobes' energy but also a shaping of the energy components away from the main-lobe at +40 dB/dec as seen in FIG. 6.

In certain embodiments, for a two-dimensional array, first order blue-noise shaping may be achieved by selecting at random between two sub-matrices [1 0; 0 1] and [0 1; 1 0] and then each randomly selected sub-matrix is used to “tile” the entire 2D array. Similarly, for a two-dimensional array, second order shaping may be achieved by selecting at random between the sub-matrices [1 0 0 1; 0 1 1 0; 0 1 1 0; 1 0 0 1] and [0 1 1 0; 1 0 0 1; 1 0 0 1; 0 1 1 0] and then tiling the entire 2D array with these random picks.

Point Scatterer Simulations

To illustrate the performance of this array, side-by-side images of the same point scatterer have been made with a full aperture array, periodic array (with every even element transmitting, and every odd element receiving), a white noise random array, and the blue noise random array, The point scatterer is located in the middle of the field of view, at the depth of 60 mm, which is the focus on transmit. Due to the introduction of the random vari-able to the random sparse arrays, the presented images are an average of 100 images from separate randomly generated sparse arrays to better illustrate the occurring phenomena. The averaging has been done prior to the log compression.

Cyst Simulations

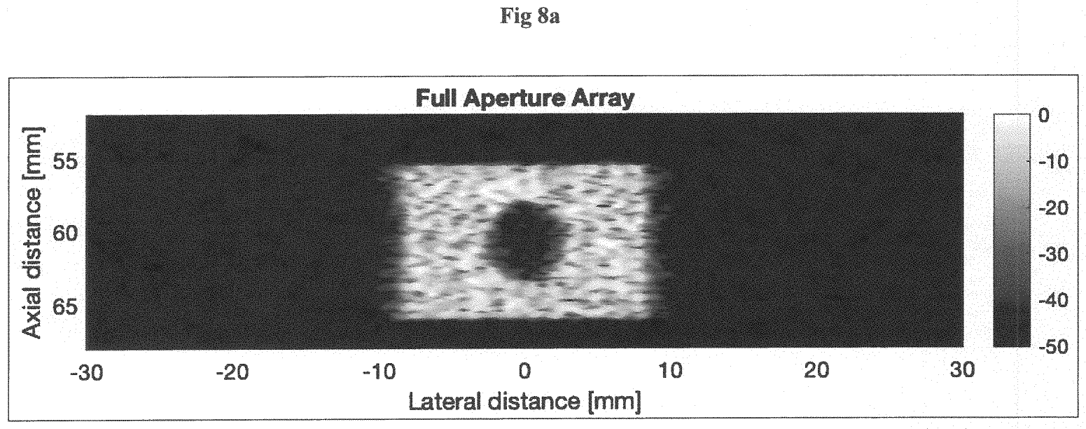

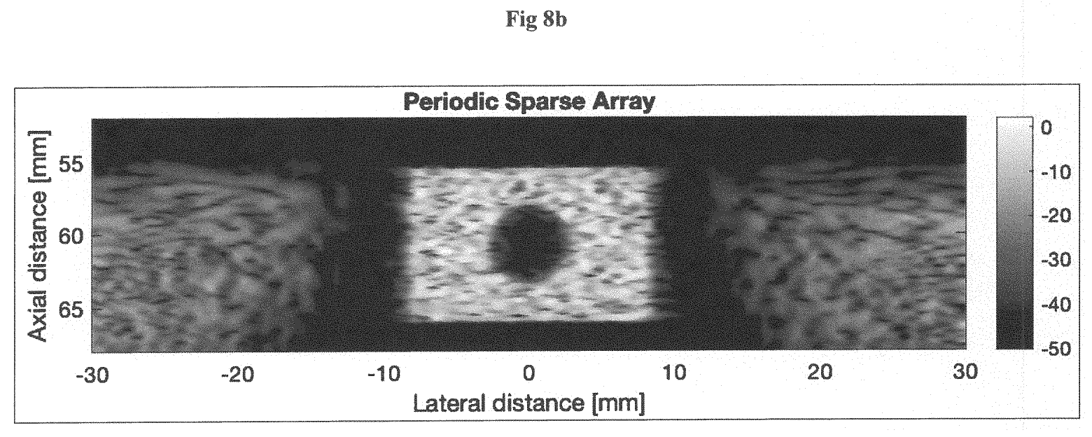

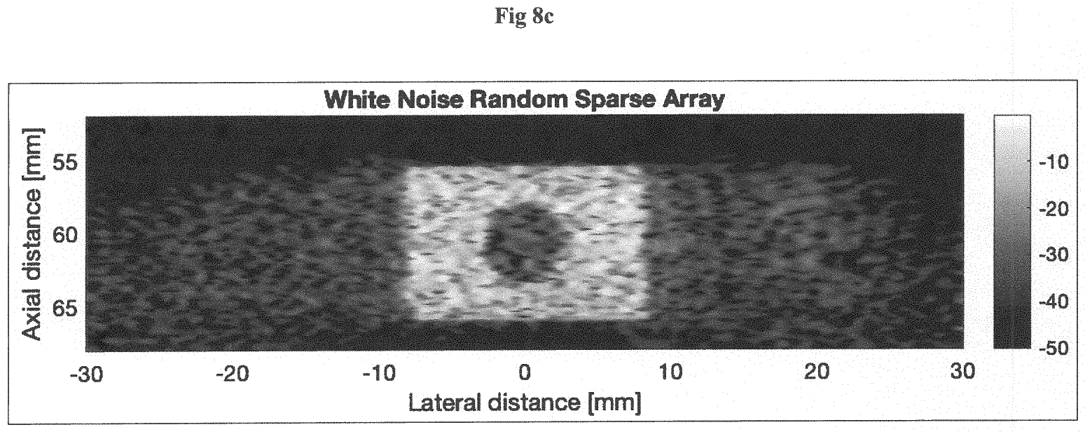

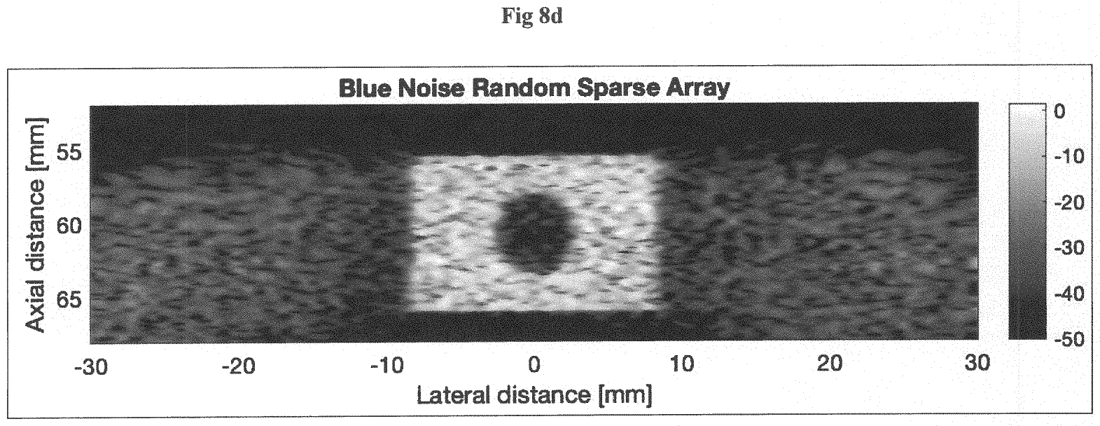

In order to further demonstrate the capabilities of the blue noise random sparse array, simulations of a cyst phantom were performed. A cyst allows standard metrics to be evaluated, such as GR, CNR, and CCNR. A 20 mm by 10 mm gel phantom was simulated, with a 3 mm hypoechoic cyst, centered around the transmit focal point, 60 mm depth. The resulting images of the full, periodic, white noise random and blue noise random apertures can be observed in FIG. 8.

The results of the measurement of CR, CNR, and GCNR for all four types of arrays can be observed in Table 1.

TABLE 1
Obtained metrics for phantom images

| | CR | CNR | GCNR |
|---|---|---|---|
| Full | 0.61 | 6.07 | 1 |
| Periodic | 0.62 | 5.95 | 1 |
| White Noise | 0.41 | 2.46 | 0.94 |
| Random | | | |
| Blue Noise Random | 0.6 | 4.91 | 1 |

Side/Grating Lobe Characterization

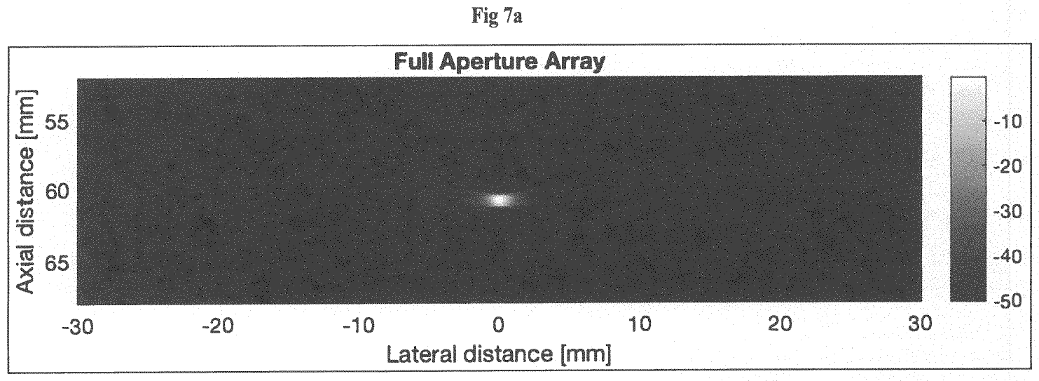

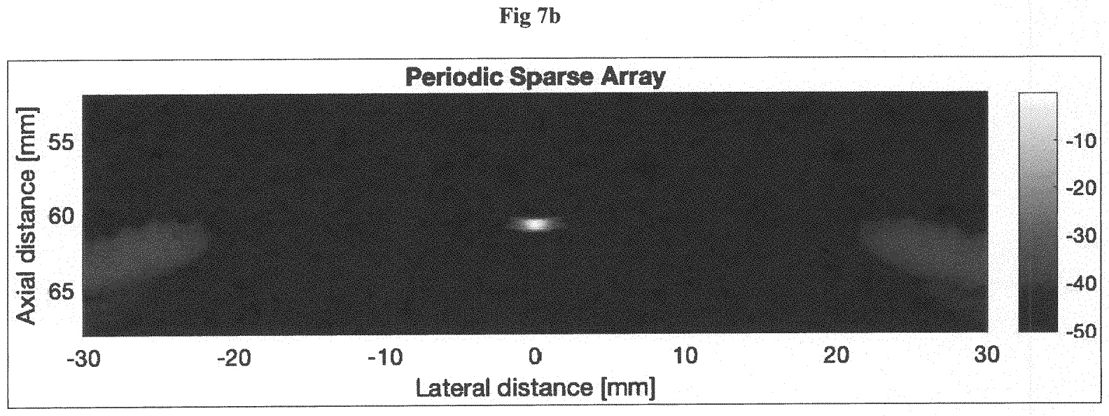

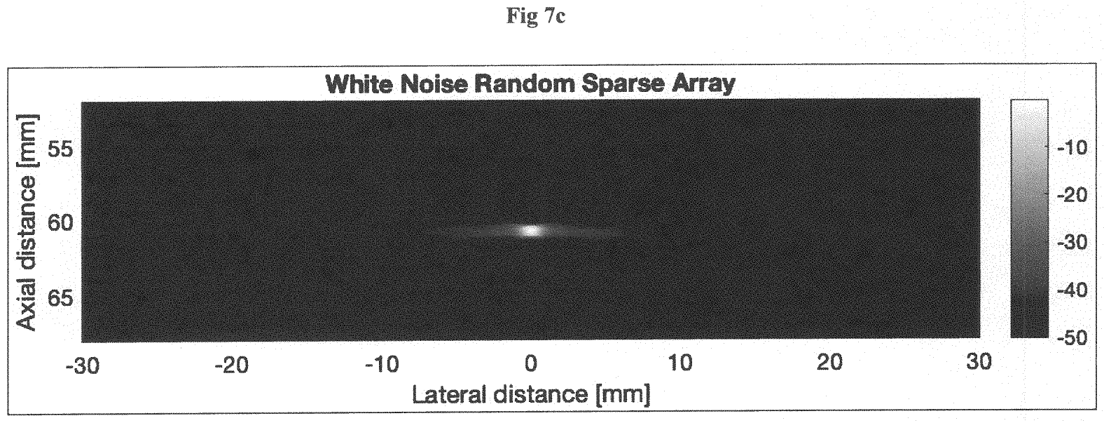

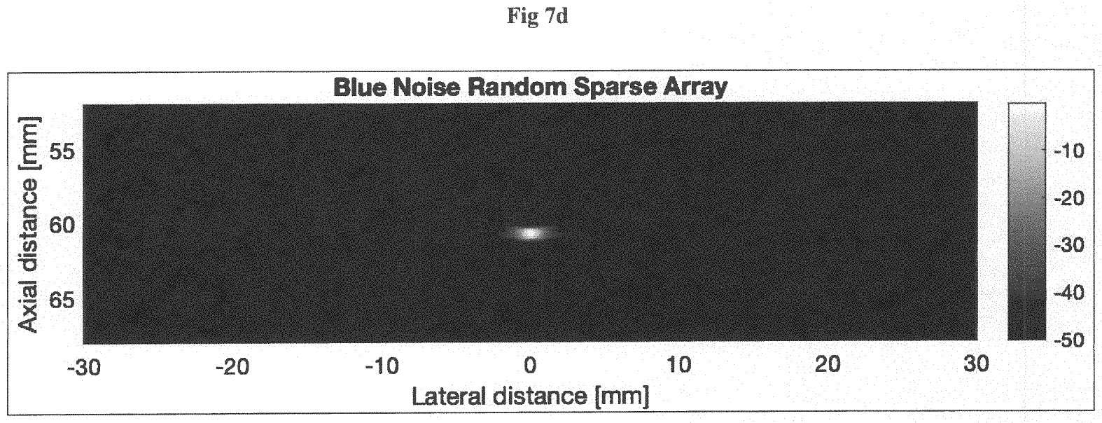

The PSF shown in FIG. 7 is evaluated in order to extract the values for the peak levels of the side and grating lobes. These peak levels are shown in Table 3.

Discussion

Due to the lowering of both delivered and the sensed power in the sparse array imaging, it was expected to obtain somewhat lower metrics as compared to the full aperture images. From the point scatterer images, by inspection, it can be seen that the periodic sparse array produces very strong grating lobes: whereas the white noise random sparse array produces very strong side lobes. Blue noise random sparse array represents a compromise solution between these two, as it has the energy spread between grating and side lobes, without putting a lot of visual accents on any of these.

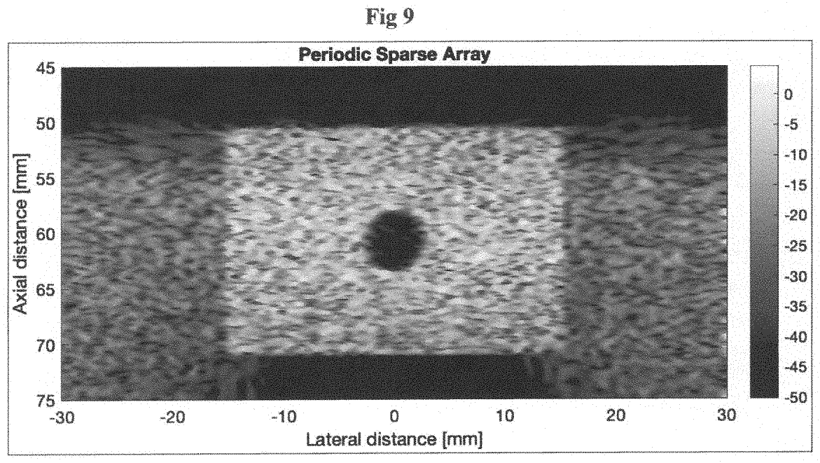

This is confirmed by the images of the cyst phantom and the quantitative measurements performed. Although the cyst remains clear with high contrast (FIG. 8, and as evident by all measurements from Table 1), because of strong grating lobes, the periodic sparse array produces strong artifacts between the phantom and the imaging medium (as observed in Table 2). This will affect images with more scatterers more severely and will make it hard to distinguish the border between the phantom and the imaging medium for the larger phantoms. An example of this can be seen in FIG. 9, where a:30 mm×20 mm phantom is imaged with a periodic sparse array.

TABLE 2
Obtained CR for near the border of the phantom

| Full | Periodic | White Noise Random | Blue Noise Random |
|---|---|---|---|
| 0.64 | 0.38 | 0.51 | 0.48 |

On the other hand, a white noise random array exhibits opposite behavior: strong side lobes render the cyst almost indistinguishable from the phantom tissue, while the border between phantom remains clear even in the larger phantoms. The blue noise random sparse array has somewhat lower contrast than the periodic sparse array, but not so significantly as the white noise random array does. However, it still shows a strong border between the phantom and the imaging medium, even for the larger phantoms. This shows the deficiency of the blue noise random sparse arrays choice in terms of image quality and provides a. significant improvement over both the white noise random and the periodic sparse array.

Furthermore, the peak side/grating lobe levels clearly show that the ar-tifact intensity varies with the array choice. From Table 3 the blue noise sparse array shows the lowest peak level, while the periodic sparse array shows the highest. Although the distribution of these lobes differs across sparse arrays, the blue noise sparse array undoubtedly introduces the lowest artifacts and, most importantly, they are not localized.

TABLE 3
Peak Side/Grating Lobe Levels (dB)

| Periodic | White Noise Random | Blue Noise Random |
|---|---|---|
| −33.93 | −38.26 | −43.77 |

The power savings of this method can be approximated by estimating the power consumption of the removed hardware. T/R switch would consume the majority of its power on the receive event. This power amounts to about 50 mW/channel (worst case) for the typical+-90V transmit voltage. Without the T/R switches, power savings can be observed in several aspects of the imaging system. First and foremost, the receiving channels would no longer consume ˜50 mW of power during the receive event. Additionally, the number of receiving channels would be smaller and is dependent on the sparse array configuration (50% of the array is receive in the proposed configuration). Finally, the number of the transmitting channels would be smaller and is dependent on the sparse array configuration, which would result in the lower amount of power necessary to send a wave through the medium.

As such, the power savings obtained through the sparse array imaging alone amount to approximately 40%, with no overhead. However, this method can be used in combination with many other power savings tech-niques developed previously. First and foremost, the sparse array imaging can be coupled with the CU to achieve an impressive 92% power savings in the imaging system hardware. However, as with a full array, it is important to mention that not every imaging scheme will benefit. from the application of CU, and particularly plane wave imaging will significantly underperform the synthetic aperture—like acquisition schemes.

Additionally, many other techniques can be used in conjunction with the proposed sparse array, and even together with the CU. For example, a 10 times power consumption reduction by offsetting the power-hungry operations to the PC and carefully designing the analog por-tion of the ultrasound system. Furthermore, an estimate of the power savings by using the compressed sensing framework. However, this estimate is provided relative to the processing time and the amount of data captured, and by no means defines any specific hardware. Nevertheless, they show a 20-fold decrease in measurement lime and a 10-fold decrease in the amount of data, at the expense of the many added components which were not discussed.

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The description herein is for the purpose of teaching the person of ordinary skill in the art how to practice the embodiments of the present application, and it is not intended to detail all those obvious modifications and variations of it which will become apparent to the skilled worker upon reading the description. It is intended, however, that all such obvious modifications and variations be included within the scope of the embodiments of the present application, which is defined by the following claims. The claims are intended to cover the components and steps in any sequence which is effective to meet the objectives there intended, unless the context specifically indicates the contrary.

What is claimed is:

1. A method of pulse-echo ultrasound imaging comprising the steps of:
separating transducer elements of an ultrasound transducer array into a first disjoint subset and a second disjoint subset,
wherein the transducer elements in the first disjoint subset perform a transmit operation only, and
wherein the transducer elements in the second disjoint subset perform an echo receive operation only; and
grouping the transducer elements into a plurality of groups of transducer elements, wherein each group consists of two or more adjacent transducer elements and wherein each group consists of at least one transducer element of the first disjoint subset and second disjoint subset into groups of transducer elements; and
randomly concatenating the groups of transducer elements into a sparse array.

2. The method of claim 1, wherein the number of the transducer element(s) of first disjoint subset equals the number of the transducer element(s) of second disjoint subset in each group.

3. The method of claim 2, wherein the first disjoint subset and the second disjoint subset in the array each produce grating-lobe(s) and side-lobe(s), and
wherein the first disjoint subset and the second disjoint subset are separated to minimize peak magnitude of each of the grating-lobe(s) and side-lobes(s).

4. The method of claim 3, wherein the array has a point spread function, wherein the point spread function has a main lobe, and
wherein the first disjoint subset and the second disjoint subset are separated to spread the energy of the side-lobe(s) away from the main-lobe of the array's point spread function.

5. The method of claim 4, wherein the spread of the energy of the side-lobe(s) reduces the energy distribution close to the main-lobe's location to nearly zero, and
wherein the energy distribution then increases at the rate of at least +20 dB/dec when moving away from the main-lobe's location.

6. The method of claim 1, wherein the groups of transducer elements comprise two or more adjacent transducer elements.

7. The method of claim 6, wherein half of the elements within a group are used for transmit, and
wherein half of the elements within a group are used for echo receive operation.

8. The method of claim 7, wherein the sparse array is formed by concatenating pairs of transducer elements selected at random from [1 0] and [0 1], wherein 1 represents an element within the pair used for transmit and 0 represents an element within the pair used for receive.

9. The method of claim 8, wherein the point spread function of the array resembles first-order blue noise.

10. The method of claim 7, wherein the sparse array is formed by concatenating quartets of transducer elements selected at random from [1 0 0 1] and [0 1 1 0], wherein 1 represents an element within the pair used for transmit and 0 represents an element within the pair used for receive.

11. The method of claim 10, wherein the point spread function of the array resembles second-order blue noise.

12. The method of claim 1, wherein the array is a one-dimensional array.

13. The method of claim 1, wherein the array is a two-dimensional array.

14. A system for pulse-echo ultrasound imaging comprising:
a sparse array of transducer elements, wherein the transducer elements of the sparse array are ordered into groups of transducer elements, and
wherein each of the groups of transducer elements has the same probability of membership in either a first disjoint subset (transmit) or a second disjoint subset (receive).

15. The system of claim 14, wherein the groups comprise two or more adjacent transducer elements, and
wherein half of the elements within a group are used for transmit, and
wherein half of the elements within a group are used for echo receive operation, and
wherein the system further comprises:
randomly concatenating the groups of transducer elements into a sparse array.

16. The system of claim 15, wherein the sparse array is formed by concatenating pairs of transducer elements selected at random from [1 0] and [0 1], wherein 1 represents an element within the pair used for transmit and 0 represents an element within the pair used for receive.

17. The system of claim 15, wherein the sparse array is formed by concatenating quartets of transducer elements selected at random from [1 0 0 1] and [0 1 1 0], wherein 1 represents an element within the pair used for transmit and 0 represents an element within the pair used for receive.

18. The system of claim 14, wherein the array is a two-dimensional array, wherein first order blue-noise shaping is achieved by selecting at random between two sub-matrices [1 0; 0 1] and [0 1; 1 0] and then tiling the array with each randomly selected sub-matrix.

19. The system of claim 14, wherein the array is a two-dimensional array, wherein second order shaping is achieved by selecting at random between the sub-matrices [1 0 0 1; 0 1 1 0; 0 1 1 0; 1 0 0 1] and [0 1 1 0; 1 0 0 1; 1 0 0 1; 0 1 1 0] and then tiling the array with each randomly selected sub-matrix.

20. The system of claim 14, wherein the number of the transducer element(s) of first disjoint subset equals the number of the transducer element(s) of second disjoint subset in each group.
